(19) United States
(12) Patent Application Publication
(10) Pub. No.: US 2022/0090992 A1
(43) Pub. Date: Mar. 24, 2022

(54) Sampler Apparatus for an Unmanned Aerial Vehicle

(71) Applicant: Terra Vigilis, Inc., Hartland, WI (US)

(72) Inventors: Michael Gordon Mortensen, Brookfield, WI (US); Charles John Luebke, Hartland, WI (US); Timothy Edward Tyre, Hartland, WI (US)

(73) Assignee: Terra Vigilis, Inc., Hartland, WI (US)

(21) Appl. No.: 17/480,386

(22) Filed: Sep. 21, 2021

Related U.S. Application Data
(60) Provisional application No. 63/081,516, filed on Sep. 22, 2020.

(51) Int. Cl. G01N 1/12; G01N 1/04; B64C 39/02; B64D 47/08

(52) U.S. Cl. CPC G01N 1/12; G01N 1/04; B64C 39/024; B64D 2211/00; B64C 2201/027; B64C 2201/12; B64D 47/08

(57) ABSTRACT

A removably attachable sampling and analysis apparatus for use with a drone which includes a housing member, a flotation member, at least two connecting members, a collapsible framework and sensors. A preferred embodiment of the present invention enables rapid deployment of the sampling and analysis apparatus to multiple target locations on a body of water to take water quality measurements and samples. The sampling and analysis apparatus can also be utilized in other applications such as soil measurements and sample collection, agricultural and forestry applications, as well as with gas sensors to measure air quality and map values in 3D space with a time stamp.

### RELATED APPLICATION

This application claims the benefit of provisional application Ser. No. 63/081,516, filed on Sep. 22, 2020, the entire contents of which are incorporated herein.

### FIELD OF THE INVENTION

This invention relates to apparatuses for sampling, and more particularly, to an apparatus for sampling which is removably connectable to an unmanned aerial vehicle or drone and has the ability to collect water and plant samples from below the surface of a body of water as well as above the surface or on land.

### BACKGROUND OF THE INVENTION

A variety of samplers have been devised to facilitate sampling of water, soil and plant material. Water quality studies of lakes, rivers, and streams presently require a significant amount of time to access the body of water with a boat and to bring the associated measurement gear, as well as the time required on the water to manually take measurements at a sequence of locations. Typically there are also limitations regarding the number of samples which can be taken from a given location as well as the problem that sampling can only occur once or maybe a few times per year. This is true for sampling of soil or vegetation as well.

One sampler device in the prior art is seen in U.S. Patent Application No. 2017/0328814 to Castendyk, et al. which discloses a sampler connected via a tether to an unmanned aerial vehicle (UAV). The tether is connected to a liquid sampling container or a probe.

A similar device is shown in EP 3 112 840 to Urbahs, et al. which also discloses a sampling device. The device in Urbahs is specifically designed to sample water surface for oil contamination using an extendible tube on an unmanned water craft (boat).

Sampling apparatuses of the prior art typically have certain disadvantages. The majority of samplers are not adapted to be removably connectable to a UAV or drone; this is a distinct disadvantage. Sampling apparatuses of the prior art are also not versatile enough to sample water, soil, plant material and other all types of matter with the same device. This is yet another disadvantage.

Some sampling apparatuses of the prior art have certain shortcomings and disadvantages to which this device is drawn. Specifically, it would be advantageous to have a sampling apparatus which is able to be removably connected to a UAV or drone and which also can sample water, soil, plant material and air thereby allowing a wide variety of samples to be efficiently taken in a desired location without requiring a significant amount of man hours.

In summary, there are problems and shortcomings in the prior art UAV samplers and it is to these needs that this device is drawn.

### SUMMARY OF THE INVENTION

The present invention includes a removably attachable sampling apparatus for use with a drone which has a housing member, flotation member, at least two connecting members, a collapsible framework and at least one removable and extendible sensor.

Preferably, the housing member has at least three sides and a central open portion. The flotation member also has at least three sides and a central open portion; the at least three sides each have a top surface and a bottom surface. It is preferable that the flotation member includes two or more cylindrical containers. The at least two connecting members each have a proximal end and a distal end and are secured to the housing member on a proximal end and secured to the flotation member on the distal end. Each connecting member is preferably equidistant from another connecting member. The collapsible framework extends from the bottom surface of the flotation member and is able to be submersible. The at least one removable and extendible sensor can extend from the bottom surface of the flotation member or from the bottom surface of the housing member.

In preferred embodiments, the cylindrical containers are for collecting samples of water or sediment. Preferably, the cylindrical containers include compressed air for water displacement when a water sample is being taken. In highly-preferred embodiments the drone is able to be detached from the sampling apparatus during underwater sampling.

Additionally, at least one sediment sampling container preferably extends from the bottom surface of the flotation member. In other preferred embodiments the sediment sampling container can also extend from the bottom surface of the housing member. The sediment sampling container is affixed to an extendible rod on a distal end and secured to a bottom surface of the flotation member on a proximal end.

In highly-preferred embodiments the at least one sensor can measure pH, turbidity, temperature and dissolved oxygen. The housing member preferably includes an ultrasonic distance sensor, a light detection and ranging sensor (“LiDAR”), a gas sensor, an accelerometer sensor, a data logger and a WIFI module. Preferred embodiments also include a solar panel, a battery, an RF antenna and a camera all mounted on or withing the housing. The housing member can send a text message to a user with sensor measurements and data from the data logger.

In preferred embodiments, the at least two connecting members are four connecting members and the framework is made of lightweight styrene. Preferred embodiments include at last one cam lock for removably coupling the drone to the housing member. Alternative embodiments include a hook on the UAV or drone and a loop securement member on the housing member for removably coupling the UAV and housing member together.

It should be noted that the terms “UAV” and “drone” are used interchangeably throughout this application. “UAV” refers to and means an unmanned aerial vehicle.

The term “payload capacity” as used herein means the gross load weight the drone is capable of safely carrying.

The term “LiDAR” as used herein means a light detection and ranging sensor.

### OBJECTS OF THE INVENTION

It is an object of this invention to provide a sampling device which is removably connectable to a UAV.

Another object of this invention is to provide a sampling device which has the ability to sample water, soil, air and plant material.

Yet another object of this invention is to provide a sampling device which has improved efficiency and minimizes the risk associated with sampling.

These and other objects of the invention will be apparent from the following descriptions and from the drawings.

### BRIEF DESCRIPTION OF THE DRAWINGS

The drawings illustrate preferred embodiments including the above-noted characteristics and features of the device. The device will be readily understood from the descriptions and drawings. In the drawings:

FIG. 1 is a perspective view of the sampling apparatus submerged in water;

FIG. 2 is a perspective view of the sampling apparatus in FIG. 1;

FIGS. 3A-4C are perspective views of embodiments of the flotation member;

FIG. 5 is a diagram of the sampling apparatus in FIG. 1 including sensors and actuators;

FIGS. 6A-6B are perspective views of the attachment devices for coupling the drone to the sampling apparatus;

FIGS. 7A-7B are cross-sectional views of the cylindrical containers;

FIG. 8A is a cross-sectional view of the sediment sampling container in an open, a collecting and a closed position;

FIG. 8B is perspective view of the sediment sampling container with a rotational actuator; and

FIG. 9 is a perspective view of the drone detached from the sampling apparatus of FIG. 1.

### DETAILED DESCRIPTION OF PREFERRED EMBODIMENTS

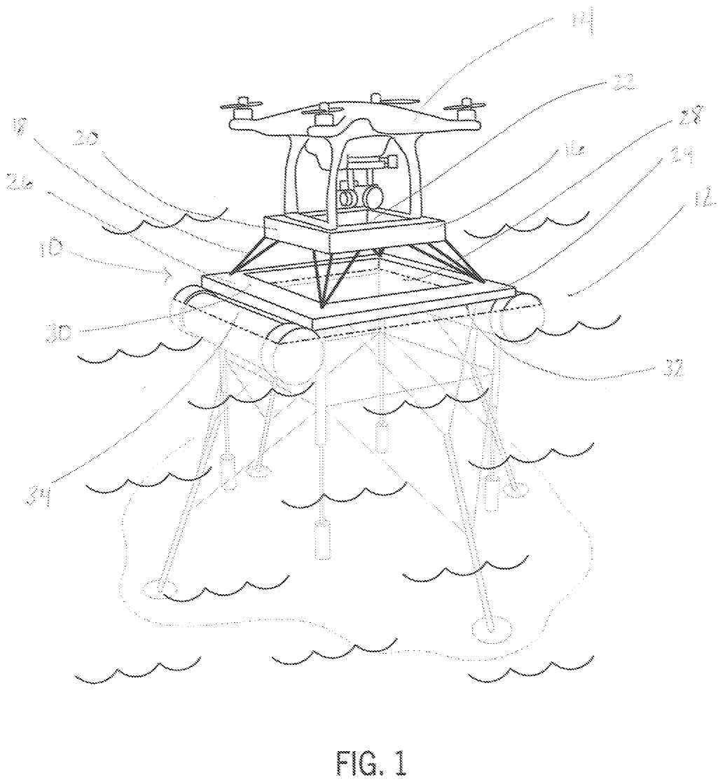

A preferred embodiment of the present invention is shown in FIGS. 1-9. The disclosed invention enables rapid deployment of a Drone Measurement System (DMS) to multiple target locations on a body of water to take water quality measurements and samples in significantly less time, and reduces the risk and time required for people to access the body of water from a shoreline to take measurements as well as having to be on the water in less than ideal conditions (i.e., cold, wind, currents, thin ice, etc.).

While the solution described in the following text and the accompanying FIGS. 1-9 largely focuses on examples of improvements utilizing a drone measurement system for use in water quality measurement and sampling applications, those skilled in the art will recognize that a drone measurement system can be used in other applications. The drone measurement system described herein can be useful also for soil measurements and sample collection as well as for agricultural and forestry applications (including vegetation and infestation studies). Through the use of a gas sensor, the drone measurement system can also be used to measure air quality and map values in 3D space with a time stamp.

Example embodiments disclosed herein are directed to a sampling apparatus for use with a drone. Example embodiments are described herein with reference to the accompanying FIGS. 1-9, however, these example embodiments are not limiting and those skilled in the art will appreciate that various modification are within the scope of this disclosure. Example embodiments can be used with the sampling apparatus disclosed herein for any of a number of applications, including but not limited to water quality, soil analysis, air quality, vegetation analysis and infestation analysis.

FIG. 1 illustrates the sampling apparatus 10 floating on the water surface 12 with a drone 14 attached and sitting on top of it. In FIG. 1, a housing member 16 and connecting members 18 are above the water level and sampling apparatus 10 is floating on the surface of the water 12. As seen in FIG. 1, housing member 16 has at least three sides 20 and a central open portion 22. A flotation member 24 also has at least three sides 26 and a central open portion 28. The at least three sides 26 of flotation member 24 also include a top surface 30 and a bottom surface 32. At least two or more cylindrical containers 34 are attached to flotation member 24.

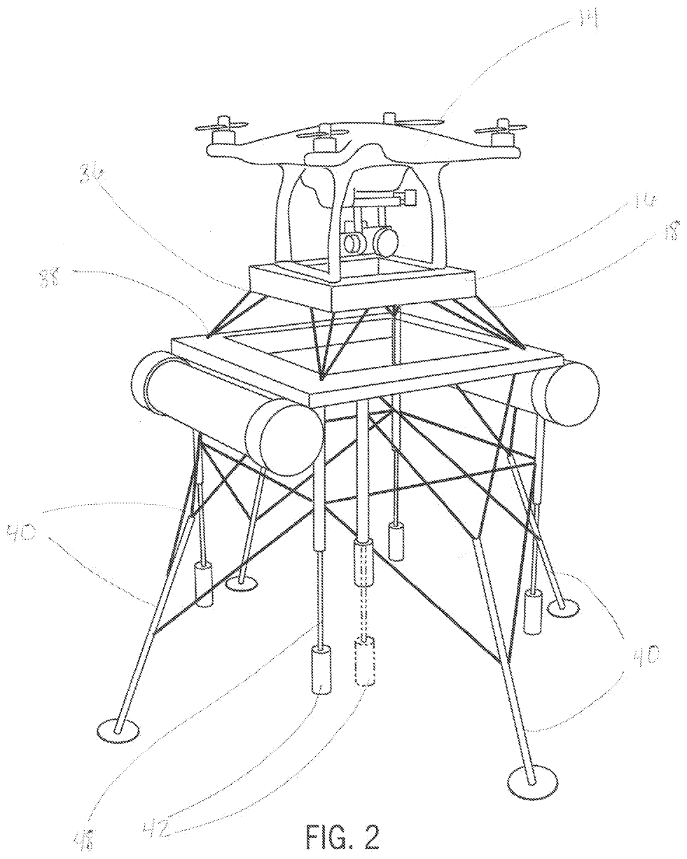

FIGS. 1-2 illustrate that sampling apparatus 10 has at least two connecting members 18 but may also have several connecting members. Connecting members 18 each have a proximal end 36 and a distal end 38. Connecting members 18 are secured to housing member 16 on proximal end 36 and secured to flotation member 24 on distal end 38, each connecting member 18 is equidistant from another connecting member 18.

A collapsible framework 40, as seen in FIGS. 1-2, extends from bottom surface 32 of flotation member 24 and is submersible (see FIG. 1). Framework 40 is made of a material such as lightweight styrene. FIGS. 1-2 illustrate that sampling apparatus 10 includes at least one removable and extendible sensor 42 and can include multiple sensors 42. Sensor 42 can also extend from housing member 16 in alternative embodiments. Sensor 42 can measure pH, turbidity, temperature and dissolved oxygen.

FIGS. 1-2 illustrate that cylindrical containers 34 are for collecting samples of water or sediment. In some embodiments, cylindrical containers 34 include compressed air for water displacement. Other embodiments include at least one sediment sampling container 46 which extends from bottom surface 32 of flotation member 24. Sediment sampling container 46 is affixed to an extendible rod 48 on a distal end and secured to bottom surface 32 of flotation member 24 on a proximal end.

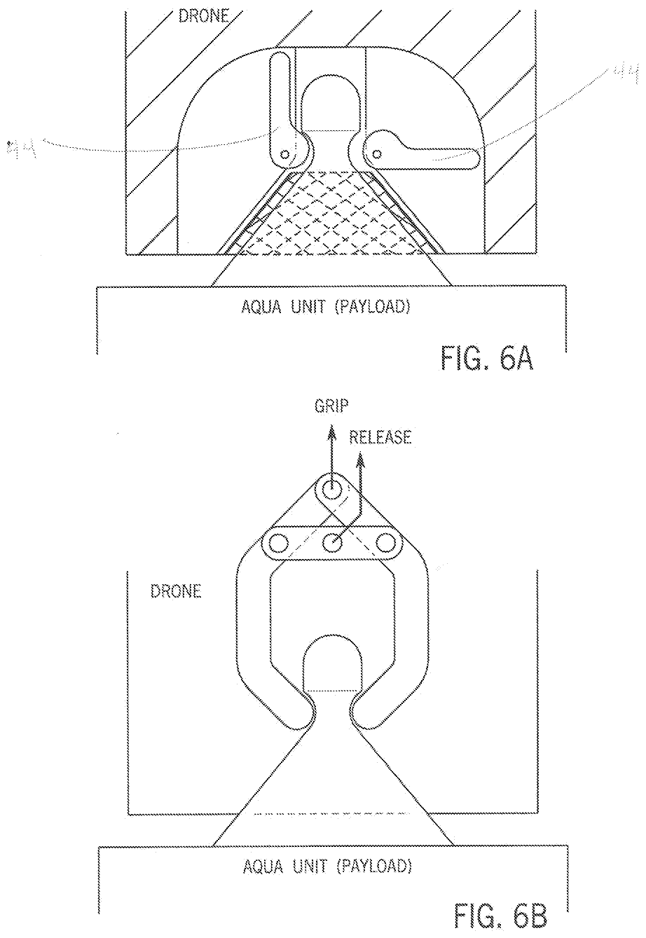

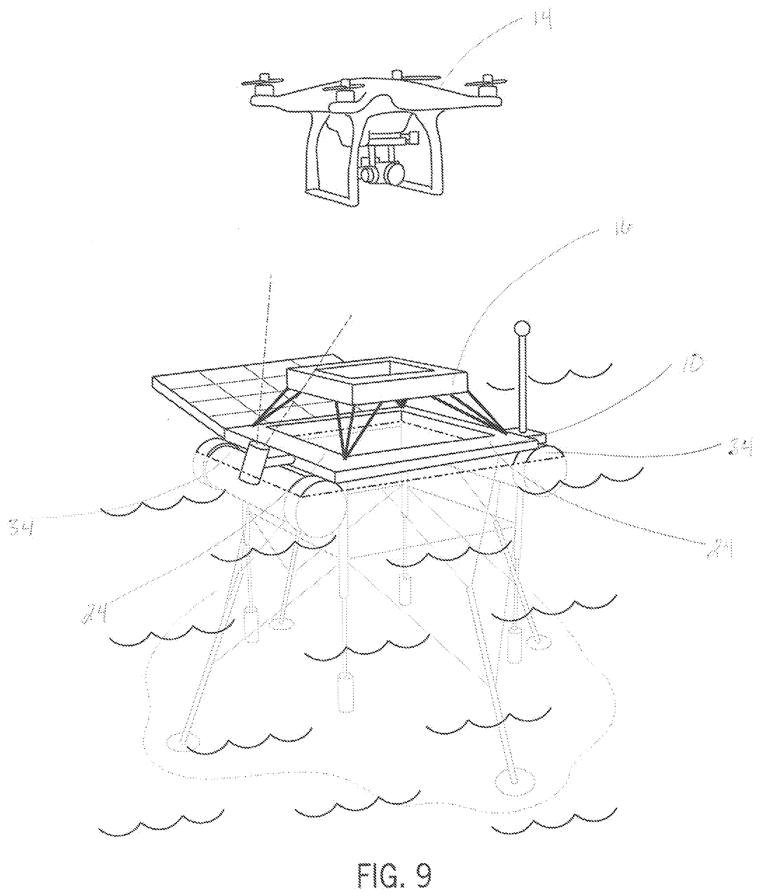

In some embodiments, drone 14 is able to be detached from sampling apparatus 10 during underwater sampling as seen in FIGS. 6A-6B and 9. As seen in FIGS. 6A, at least one cam lock 44 is needed for coupling drone 14 to housing member 16.

Housing member 16 can include an ultrasonic distance sensor, a LiDAR distance sensor, a gas sensor, an accelerometer sensor, a data logger and a WIFI module. Housing member 16 has the ability to send a text message to a user with sensor measurements and data from the data logger. Sampling apparatus 10 can also include a solar panel and a battery as well as a camera and an RF antenna in some embodiments.

FIG. 2 illustrates sampling apparatus 10 removably connected to a drone 14. Sampling apparatus 10 as seen in FIG. 2 includes a lightweight framework 40, flotation member 24, housing member 16 which contains the electronics, and one or more external sensors 42. The lightweight framework 40 is collapsible for ease in transportation to and from a measurement site. Framework 40 provides legs as a base to prevent sensors 42 from inadvertently touching the bottom of a body of water. The feet on the legs are shaped to provide a larger landing surface to minimize sinking on soft sedimentary lake bottoms. A lightweight mesh screen can also be wrapped across/between the legs to provide a larger landing surface area as well as to prevent entanglement with aquatic plants or other underwater structures.

Sensors 42 and sampling vials such as cylindrical containers 34 or sediment sampling container 46, can be lowered/extended by an actuator to perform measurements at different depths and to do soil measurements and sampling. The legs of framework 40 provide stability when on the surface and underwater and assist sampling apparatus 10 with staying in an upright position. Fins or an extendable curtain on framework 40 act like a storm anchor so sampling apparatus 10 does not drift from the desired location due to wind and waves impinging on the cross-sectional area above the water. Drone 14 can also maintain the GPS location while sampling apparatus 10 is floating on the water.

Sampling apparatus 10 is not heavy (preferably under 1 lb.) and can be attached, carried, and flown using any commercially available “off the shelf” drone with sufficient payload capacity. Drone 14 can fly with sampling apparatus 10 to specific lake locations and float on the surface of the water to conserve drone battery power while sampling apparatus 10 takes measurements with the onboard sensors 42. The measurements taken are logged within sampling apparatus 10, however, measurements can also be sent wirelessly (e.g., via text messaging) to the drone pilot. Return visits can also be made to the same GPS coordinates for direct data comparison over time.

Drone 14 can also have a payload release/attachment mechanism. For example, FIG. 6A illustrates a self-aligning ball and cam coupler 44. Any securement device can be used and other examples are a pintle hook (not shown) or as seen in FIG. 6B a multi-finger clamp/gripper/claw which can detach from sampling apparatus 10 (payload unit) while landed to provide extended data collections and analysis on the surface or to enable sampling apparatus 10 to submerge. Some embodiments have a fixed (non-swiveling) coupler between drone 14 and sampling apparatus 10 while airborne for stable horizontal maneuvering.

However, if the payload mass is more than the mass of the drone, it may be advantageous to have a gimbled coupler, ball, or short tether (loop) to enable the drone to pitch and yaw independent of the sampling apparatus below it while still minimizing sway. The self-alignment docking of the drone with the sampling apparatus (payload) can be achieved with a tapered mesh cone on the drone. The mesh cone can be made with a low friction plastic to glide/slide the ball into place, and also provides low weight and low cross- sectional area during flight. The cam and gripper mechanisms provide a positive lock between the drone and sampling apparatus when the actuator is in the closed position.

Sampling apparatus 10 can intake water into the side flotation canisters, also referred to herein as the cylindrical containers 34, allowing the system to dive and utilize a pressure transducer to determine the descent to a specified depth. Sampling apparatus 10 would also have external lighting and cameras to enable viewing of subsurface wave action, aquatic plants, aquatic life, suspended particulates and water quality, and sedimentation.

While detached from the drone and/or submerged, sampling apparatus 10 is fully automated. After taking the programmed and/or commanded measurements, and/or after a given amount of time, and/or detecting a low-battery power level, sampling apparatus 10 will release compressed air into the canisters allowing the unit to surface. This enables the drone to “dock” and re-engage with sampling apparatus 10 to safely and securely attach and lock sampling apparatus 10 to the drone before taking flight.

As an alternative embodiment, actuators can be used to lower one or more sensors to desired depths while sampling apparatus 10 stays floating on the water surface. Some embodiments include a solar panel mounted on the top of sampling apparatus 10 which enables the batteries to be recharged while on the water surface thus, enabling data collection and analysis to be extended indefinitely, for radio broadcast of the data to a remote receiver, and to receive new commands or programs via radio from a remote transmitter.

Water and/or soil samples can also be collected while sampling apparatus 10 is on or under the water surface. This permits post analysis in a lab of additional water quality parameters, including but without limitation for phosphorus, chlorophyll, or other compounds, contaminants or chemicals.

The primary sensors of sampling apparatus 10 include but are not limited to pH, temperature, turbidity and dissolved oxygen. Sampling apparatus 10 can also have a GPS module and a multi-axis accelerometer to measure wave activity while floating on the water surface. The drone with sampling apparatus 10 can also fly above waves and use the LiDAR and ultrasonic (acoustic) sensor to measure wave heights. Sampling apparatus 10 can carry a pressure sensor to measure depth as well as wave activity from under the water surface.

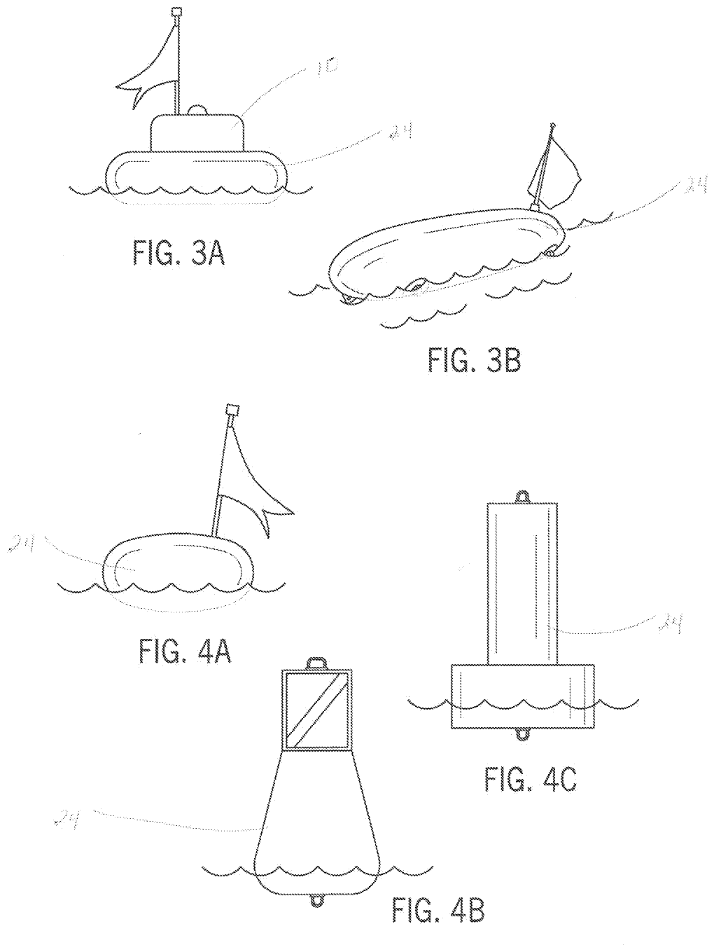

Alternative embodiments of flotation member 24 are illustrated in FIG. 3A and include a circular-shaped flotation member 24 with an open aperture in the middle with sampling apparatus 10 secured in the middle. This configuration provides a landing platform and attachment for the drone. The diameter of the circular-shaped flotation member 24 is different that the diameter of the drone rotors, thus, facilitating air flow during flight with minimal interferenc e. In another embodiment as shown in FIG. 3B, a torpedo-shaped flotation member 24 can be used with sampling apparatus 10 secured in the middle. This configuration also provides a landing platform and attachment for the drone. The size of the torpedo-shaped flotation member is different that the diameter of the drone rotors, thus, facilitating vertical air flow for lift with minimal interference during flight. In some embodiments, housing member 16, which includes the electronics, is completely contained within a single flotation member 24.

FIG. 4A illustrates that flotation member 24 may also be a round buoy with a flat top for the drone to land upon or with a loop to release and retrieve sampling apparatus 10. FIG. 4B illustrates an inflatable scuba buoy with a loop on top for the drone to release and retrieve sampling apparatus 10. FIG. 4C is a cylindrical regulatory buoy with sampling apparatus 10 located inside with a semi-rigid (cable) handle loop(s) on top for the drone to use to release and retrieve sampling apparatus 10.

Regulatory buoys are white with a single orange band at the top and bottom of the exposed buoy. An Informational regulatory buoy is signified by an open orange rectangle symbol spaced between these bands with wording or message in black letters. The diameter of the flotation device should be smaller than the diameter of the drone rotors to minimize interference of airflow during flight.

In yet another embodiment (not shown), the flotation device can be made from short sections of a foam swim “noodle”. These alternative embodiments also include Coast Guard recognized informational markings on sampling apparatus 10 or an

Alpha flag to provide enhanced visibility in high traffic areas or navigable waterways. Sampling apparatus 10 may also include a light for illumination at night. These alternative embodiments can also respond to wave action differently based on their shape, buoyancy, and stability which influences the wave measurement method used.

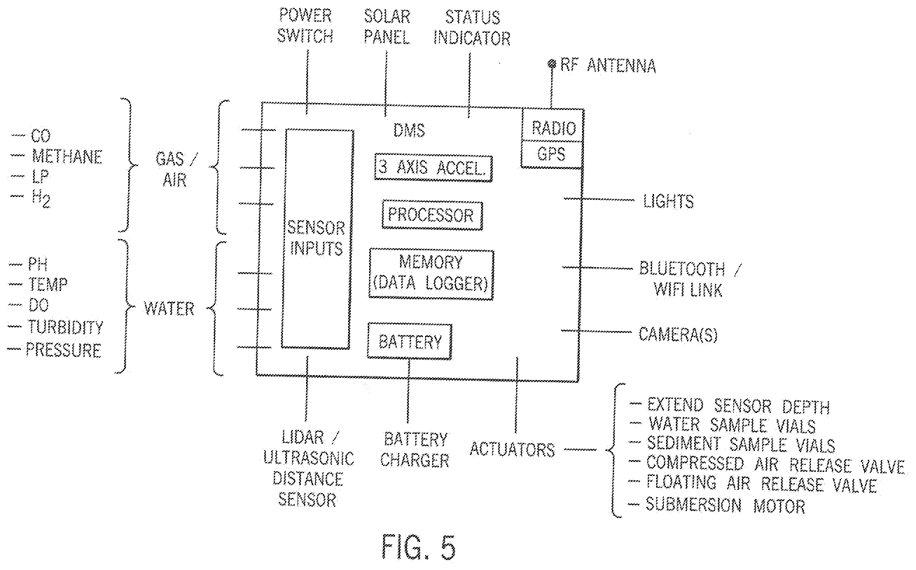

FIG. 5 is a block diagram of sampling apparatus 10 including external sensors 42 and actuators on sampling apparatus 10. Sampling apparatus 10 is housed within a sealed enclosure to protect it from water at the surface as well as for submersion. Sealed connectors (preferably circular with 0-rings) are used to connect external sensors, actuators, lights, etc.

A processor with an associated program executes a series of commands/instructions to collect sensor data at specific time intervals and save it to memory (data logger) for later retrieval. When on the water surface, sampling apparatus 10 can transmit measured data via radio to a remote receiver. When sampling apparatus 10 returns to the operator, the data in memory can also be retrieved with a removal memory device (i.e., micro SD card, USB, etc.). When on the water, surface sampling apparatus 10 can also receive commands from a remote transmitter. An internal GPS can determine the present and past positions of sampling apparatus 10 so that data can be correlated to a specific location. Sampling apparatus 10 is able to transmit its status and location for retrieval by the drone.

Sensor inputs for water quality include but are not limited to pH, temperature, Dissolved Oxygen (DO) and turbidity. Wave measurements can be made with various sensors including but not limited to pressure, LiDAR and ultrasonic. A 3-Axis accelerometer internal to sampling apparatus 10 can also provide a way to measure waves. Gas sensors can also be included but not limited to CO, H2, Methane and LP in order to gather specific air quality data when airborne or sitting at a location on land, a building, or on the water.

Additional external connections on sampling apparatus 10 can provide for external cameras, lights, actuators, battery packs, solar panels, propulsion, etc. External cameras can be used to take photos of water quality, aquatic plants, sedimentation, etc., as well as videos of subsurface wave action (with external streamers or vegetation to detect/measure motion). A camera with image processing and a Secchi disc on an adjustable, calibrated tether can automate measurements for water transparency/turbidity. The cameras can be self-contained (i.e., GoPro) and receive wireless commands from sampling apparatus 10, or it can be connected to sampling apparatus 10 to receive wired commands and/or battery power.

External lights can be used to provide illumination underwater for the cameras, particularly at increased depths with reduced lighting or at night. An external light can also provide illumination so sampling apparatus 10 can act as a visible navigation buoy at night. External connections are also provided to power and control external actuators including but not limited to spool for extending tethered sensors to increase depth of sensing location, open and close water sample vials, open and close sediment sample vials, actuate a valve on the flotation air chamber to release air and allow sampling apparatus 10 to submerge, actuate a valve on a compressed air chamber to release compressed air into the flotation air chamber to allow sampling apparatus 10 to resurface.

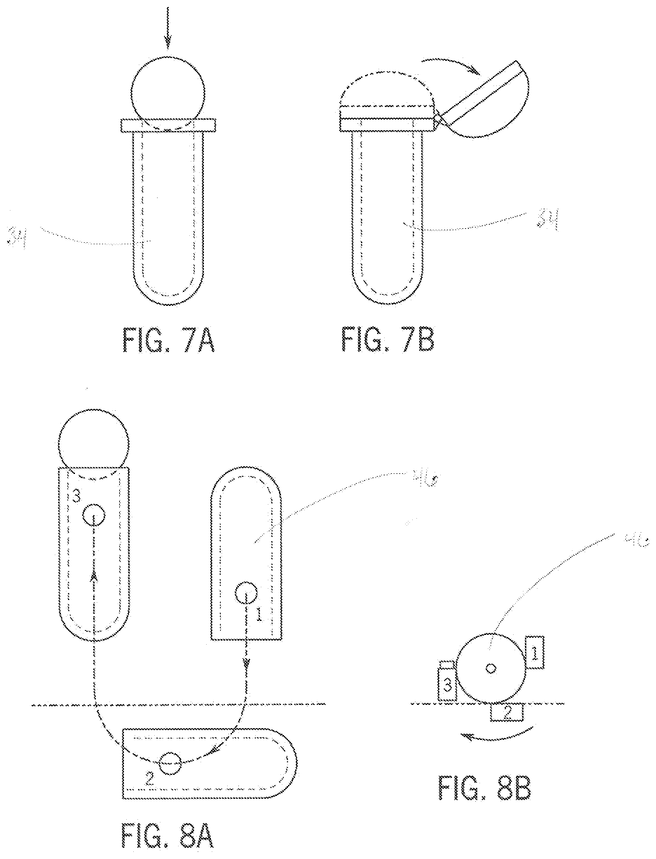

FIG. 7A illustrates a water sample vial such as cylindrical containers 34 that can be opened and closed with a ball-shaped plug using a linear actuator. FIG. 7B illustrates s a water sample vial that can be opened and closed with a hinged cover lid (horizontally or vertically) using a rotary actuator. FIGS. 8A and 8B show a sediment sampling container 46 (or corer) that can be located on a rotational actuator with multiple positions for open, collect (scoop), and close (with cover).

To minimize weight of sampling apparatus 10 so that it can be more easily transported by drone at lower power levels, the compress air chamber is preferably filled before the drone carries sampling apparatus 10 to its destination (i.e., no compressor onboard sampling apparatus 10).

A battery may be internal to the sealed sampling apparatus 10, or an external sealed battery pack may be connected to sampling apparatus 10 to allow for swapping of battery packs for quick redeployment of sampling apparatus 10. A solar panel mounted on sampling apparatus 10 can also be connected to provide recharging on the water surface for extended operation. One or more propulsion motors can be added to sampling apparatus 10 to enable the unit to submerge vertically more quickly. Ancillary inputs and outputs on sampling apparatus 10 include but are not limited to a power switch and status indicators/displays.

The sediment sampling container 46 illustrated in FIG. 8 is attached at the end of a rod (similar to the pH, turbidity, temperature or DO sensors). The rod is an extendable rod with an extendable actuator for easy access to reach the sediment below the legs when sitting on the bottom of a body of water. An actuator (not shown) would also be used to retrieve the sediment sample and preferably to close the vial. The water sample vials or cylindrical containers 34 can be located anywhere on sampling apparatus 10 that would be below the water line when floating.

FIG. 9 illustrates that the drone 14 is able to detach from sampling apparatus 10 during sampling. Also, FIG. 9 illustrates that sampling apparatus can include features such as a solar panel, camera and RF antenna.

Although the inventions are described with reference to example embodiments, it should be appreciated by those skilled in the art that various modifications are well within the scope of the invention. Those skilled in the art will appreciate that the present device is not limited to any specifically discussed application and that the embodiments described herein are illustrative and not restrictive.

A wide variety of materials are available for the various parts discussed and illustrated herein. Although the device has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. A removably attachable sampling apparatus for use with a drone comprising:
a housing member including at least three sides and having a central open portion;
a flotation member including at least three sides and having a central open portion, the at least three sides each having a top surface and a bottom surface, the flotation member including two or more cylindrical containers;
at least two connecting members, the connecting members each having a proximal end and a distal end, the connecting members secured to the housing member on a proximal end and secured to the flotation member on the distal end, each connecting member being equidistant from another connecting member;
a collapsible framework extending from the bottom surface of the flotation member and being submersible; and
at least one removable and extendible sensor.

2. The sampling apparatus of claim 1 wherein the at least one sensor extends from the bottom surface of the flotation member or from the bottom surface of the housing member.

3. The sampling apparatus of claim 1 wherein the cylindrical containers are for collecting samples of water or sediment.

4. The sampling apparatus of claim 3 wherein the cylindrical containers include compressed air for water displacement.

5. The sampling apparatus of claim 1 wherein the drone is able to be detached from the sampling apparatus during underwater sampling.

6. The sampling apparatus of claim 1 wherein the at least one sensor can measure pH, turbidity, temperature and dissolved oxygen.

7. The sampling apparatus of claim 1 wherein the at least two connecting members are four connecting members.

8. The sampling apparatus of claim 1 wherein the housing member includes an ultrasonic distance sensor, a LiDAR distance sensor, a gas sensor, an accelerometer sensor, a data logger and a WIFI module.

9. The sampling apparatus of claim 1 wherein the framework is lightweight styrene.

10. The sampling apparatus of claim 1 further including a solar panel and a battery.

11. The sampling apparatus of claim 1 further including a camera and an RF antenna.

12. The sampling apparatus of claim 1 further including at least one cam lock for coupling the drone to the housing member.

13. The sampling apparatus of claim 1 wherein the housing member can send a text message to a user with sensor measurements and data from the data logger.

14. The sampling apparatus of claim 1 wherein at least one sediment sampling container extends from the bottom surface of the flotation member, the sediment sampling container being affixed to an extendible rod on a distal end and secured to a bottom surface of the flotation member on a proximal end.

15. A removably attachable sampling apparatus for use with a drone comprising:
a housing member including four sides and having a central open portion;
a flotation member including four sides and having a central open portion, the four sides each having a top surface and a bottom surface;
at least two connecting members, the connecting members each having a proximal end and a distal end, the connecting members secured to the housing member on a proximal end and secured to the flotation member on the distal end, each bracing member being equidistant from another bracing member;
a collapsible framework extending from the bottom surface of the flotation member and being submersible; and
at least one removable and extendible sensor extending from the bottom surface of the housing member or the flotation member.

16. The sampling apparatus of claim 15 wherein the flotation member includes two or more cylindrical containers.
